(19) United States
(12) Patent Application Publication
(10) Pub. No.: US 2009/0238812 A1
(43) Pub. Date: Sep. 24, 2009

(54) Method for the early indentification and prediction of an abrupt reduction in kidney function in a patient undergoing cardiothoracic surgery

(75) Inventors: Cormac Gerald Kilty, County Dublin (IE); Claire Victoria McGrath, County Dublin (IE); Patrick Thomas Murray, Chicago, IL (US); Kerstin Schuster, County Dublin (IE); Jay Lawrence Koyner, Chicago, IL (US)

Correspondence Address: BIRCH STEWART KOLASCH & BIRCH, PO BOX 747, FALLS CHURCH, VA, 22040-0747, US

(73) Assignee: BIOTRIN INTELLECTUAL PROPERTIES LIMITED

(21) Appl. No.: 12/076,411

(22) Filed: Mar. 18, 2008

(51) Int. Cl. A61K 38/45; G01N 33/573; A61P 13/12

(52) U.S. Cl. 424/94.5; 436/501; 435/7.4

(57) ABSTRACT

A method for the early identification and prediction of an abrupt reduction in kidney function in a patient undergoing cardiothoracic (CT) surgery, including Cardio-Pulmonary Bypass (CPB), comprises contacting a urine sample from the patient with a capture molecule for a biomarker, especially πGST, specific for the distal region of the renal tubule and which biomarker is released from said region when there is damage to said region indicative and predictive of an abrupt reduction in kidney function, the biomarker being detectable as early as intraoperatively or in the recovery stage post CT surgery, for example prior to transfer of the patient to the Intensive Care Unit (ICU), allowing for immediate corrective medical intervention. The method can be used to detect Acute Kidney Injury (AKI) and a requirement for Renal Replacement Therapy (RRT) namely dialysis, earlier than two hours post CT surgery and as early as zero hours post or during CT surgery or CPB.

### TECHNICAL FIELD

This invention relates to the early identification and prediction of an abrupt reduction in kidney function in a patient undergoing cardiothoracic surgery as a result of renal ischemia and, in particular, to a biomarker for the detection thereof.

### BACKGROUND ART

An abrupt reduction in kidney function occurs frequently following cardiothoracic (CT) surgery. Thus, Acute Kidney Injury (AKI) is common following CT surgery occurring in 7-42% of patients (Mora Mangano, C. et al (1998) Ann Intern Med 128:194-203; and Tuttle, K. R. et al (2003) Amer J. Kid Dis 41:76-83.) Small changes in serum creatinine have been shown to correlate with increased morbidity and mortality, following CT surgery (Lassnigg, A. et al (2004) J. Am Soc Nephrol 15;1597-1605)

Measurement of creatinine is the standard test in the clinic for measuring kidney function. If kidney function is abnormal, creatinine levels will increase in the blood due to decreased excretion of creatinine in the urine. Creatinine levels vary according to a person's age, size and muscle mass. In acute conditions build up of creatinine in the blood may take up to 24-72 hours to occur.

Patients who develop severe AKI requiring Renal Replacement Therapy (RRT), after CT surgery have a greatly increased in-hospital mortality (63%) compared to those with non-dialyzed AKI (19%), or stable renal function (0.9%) (Mora Mangano, C. et al (1998) supra).

Koyner, J. L. et al (poster presentation at American Society of Nephrology, Renal Week 2007, Oct. 31-Nov. 5, 2007, Moscone Center, San Francisco, California) have investigated urinary Cystatin C (CyC) and Neutrophil Gelatinase-Associated Lipocalin (NGAL) in patients with AKI following adult cardiac surgery. Koyner, J L et al found that urinary CyC excretion increases in the early post-operative period following adult CT surgery and concluded that urinary CyC may be a useful early biomarker for the development of AKI as it appears to correlate with the severity of AKI and thus the future need of RRT. Similarly, Koyner, J L et al found that urinary NGAL in the early post-operative period appears to predict the development of AKI and correlate strongly with the future need of RRT.

U.S. Publication 2004/0219603 discloses that urinary NGAL measured within two hours of cardiac surgery was predictive of Acute Renal Failure (ARF) as reflected by serum creatinine peak, which occurs several hours or even days later.

Koyner J. L. et al (2007) (supra) show that for both CyC and NGAL the main increase occurs in the ICU (Intensive Care Unit) phase post CT surgery.

Eijkenboom, J. J. A. et al (2005) Intensive Care Med 31:664-667 show that an increase in GST excretion following cardiac surgery was not correlated with changes in plasma creatinine and is not associated with clinically relevant renal injury.

Davis, C. L. et al (1999) J Am Soc Nephrol 10:2396-2402 discloses that urinary GST excretion increased in most patients after CPB, however, this increase was not associated with the development of clinically apparent ARF.

There is a need for a biomarker which predicts the development of AKI at the earliest stage post CT surgery, ideally at zero hours in the recovery room and prior to transfer to ICU or earlier, namely intraoperatively, so as to enable corrective action to be taken as soon as possible for those patients who develop AKI with the attendant consequences.

Currently no drug therapy is available for counteracting the effects of an abrupt reduction in kidney function as seen in post CT surgery. Accordingly, the surgeon and other attending medical professionals will endeavour to reduce the effects of renal ischemia by managing fluid levels and other physiological parameters. However, as indicated above, frequently, if such measures do not prove successful, the patient will require RRT, namely dialysis.

### DISCLOSURE OF THE INVENTION

A method for the early identification and prediction of an abrupt reduction in kidney function in a patient undergoing cardiothoracic (CT) surgery, which method comprises contacting a urine sample from the patient with a capture molecule for a biomarker specific for the distal region of the renal tubule and which biomarker is released from said region when there is damage to said region indicative and predictive of an abrupt reduction in kidney function, the biomarker being detectable as early as intraoperatively, allowing for immediate corrective medical intervention.

The method according to the invention, by providing a means of detecting damage to, and predicting the extent of damage to, the kidney as early as intraoperatively represents a very significant advance in the management and treatment of patients undergoing CT surgery.

By “capture molecule” herein is meant any molecule or portion thereof which binds reversibly or irreversibly to said biomarker, so that said biomarker can be detected in the urine sample.

According to one embodiment of the invention, the biomarker is detectable in the recovery stage post CT surgery.

The method according to the invention, by providing a means of detecting damage to, and predicting the extent of damage to, the kidney in the recovery stage post CT surgery, allows for the appropriate medical intervention to be taken, dependent on the level of the biomarker detected during the recovery stage or earlier, namely intraoperatively.

Thus, the method according to the invention can indicate and/or predict a reduction in kidney function significantly earlier than the current standard creatinine test or other current methods hereinabove mentioned.

Preferably, the biomarker is detectable prior to transfer of the patient to the Intensive Care Unit (ICU).

According to one embodiment of the invention, the abrupt reduction in kidney function is caused by Acute Kidney Injury (AKI).

In such a situation, the reduction in kidney function can be reversed by managing fluid levels and other physiological parameters.

According to an alternative embodiment, the abrupt reduction in kidney function results in a requirement for Renal Replacement Therapy (RRT).

In such a situation, the RRT will generally involve putting the patient on dialysis supplemented, as required, by managing fluid levels and other physiological parameters. However, RRT includes peritoneal dialyses, hemofiltration, renal transplantation, depending on the severity of the renal damage.

Thus, it will be appreciated that use of the method according to the invention can result in a significant reduction of the deleterious side effects of renal ischemia post CT surgery.

Preferably, the biomarker is detected earlier than 2 hours post CT surgery or earlier than two hours post Cardio-Pulmonary Bypass (CPB).

Further, preferably, the biomarker is detected at zero hours post CT surgery or CPB.

Preferably, the biomarker is pi glutathione S transferase (πGST), also referred to hereinafter as pi GST.

According to one embodiment, the biomarker is detected by immunoassay.

When the biomarker is πGST, the capture molecule is preferably an antibody to πGST. The antibody may be a monoclonal or a polyclonal antibody which binds to πGST.

For example, the biomarker πGST can be detected using an enzyme immunoassay, more particularly an Enzyme Linked Immunosorbent Assay (ELISA). In this regard, the πGST can be assayed using a commercially available kit marketed by Biotrin International Limited, Dublin, Ireland as PI GST EIA, (Catalogue No. BIO 85) which is a 96 well EIA assay format kit. However, any other conventional assay for detecting πGST can be used.

It will be appreciated that when the biomarker is πGST, an enzyme, then the capture molecule therefor can also be a substrate or co-factor therefor.

Accordingly, according to a further embodiment of the invention, the biomarker can be detected enzymatically.

According to one embodiment of the invention the biomarker is detected by a point-of-care assay.

A point-of-care assay will typically be performed on a urine sample of less than 500 μl, typically 10 μl or less. In a point-of-care assay in accordance with the invention, the capture medium will be suitably a dip-stick or like device having the capture molecule affixed thereto.

The invention also provides πGST for use as a biomarker for the early identification and prediction of an abrupt reduction in kidney function in a patient undergoing CT surgery.

According to one embodiment πGST is used as a biomarker for AKI post CPB.

According to an alternative embodiment, πGST is used as a biomarker for the early identification and prediction of patients undergoing CPB requiring RRT.

It will be appreciated that individuals have different urinary biomarker reference baseline levels. Therefore, post-operative or post-treatment results should be considered in relation to the patient's pre-operative or pretreatment reference baseline biomarker level, as appropriate.

### BRIEF DESCRIPTION OF THE DRAWINGS

FIG. 1 is a graph of % change in Serum Creatinine (SCr) concentration from baseline versus time as described in Example 1;

FIG. 2. is a graph of absolute change in SCr concentration (mg/dl) from baseline versus time as described in Example 1;

FIG. 3. is a graph of πGST concentration (ng/ml) versus time as described in Example 1;

FIG. 4. is a graph of πGST concentration (ng/ml) versus time as described in Example 2;

FIG. 5. is a graph of SCr concentration as % of baseline value versus time as described in Example 2; and

FIG. 6. is a graph of absolute change in SCr concentration from baseline (mg/dl) versus time as described in Example 2.

### MODES FOR CARRYING OUT THE INVENTION

The invention will be further illustrated by the following Examples

### EXAMPLE 1

Use of πGST as a Biomarker for AKI in Patients Undergoing CT Surgery.

A retrospective study of 68 patients who had undergone elective CT surgery at the University of Chicago Hospital was carried out.

The patients were screened and approached for enrollment. The patients were excluded if they met any of the following criteria:
Pre-existing End Stage Renal Disease (ESRD) (on RRT) or Renal Transplant.
Age<18 years old.
Use of radiocontrast within 24 hours of surgery.
Change in thyroid hormone replacement dose in the last 2 weeks
Change in thyroid chronic corticosteroids dose in the last 2 weeks
Unstable renal function (Δ Serum Creatinine ≧0.2 mg/dl in the last 2 months of Oliguria defined as <400 ml/day).

Urine and blood samples were collected and stored.

The urine samples were tested for the presence of πGST using the aforementioned πGST EIA available from Biotrin International Limited (Catalogue Number BIO85).

Serum Creatinine (SCr) was measured using the Jaffé Method in a manner known per se on a Beckman Unicel DxC 600 autoanalyser (Beckman Coulter, Fullerton, Calif., USA).

AKI was determined by change in SCr as defined as:

An abrupt (within 48 hours) reduction in kidney function currently defined as

1) absolute increase in serum creatinine of more than or equal to 0.3 mg/dl (≧26.4 μmol/l); or

2) a percentage increase in serum creatinine of more than or equal to 50% (1.5-fold from baseline).

This definition is consistent with the usual definition used, for example, by Mehta, R. L., et al (2007) Critical Care; 11: R31

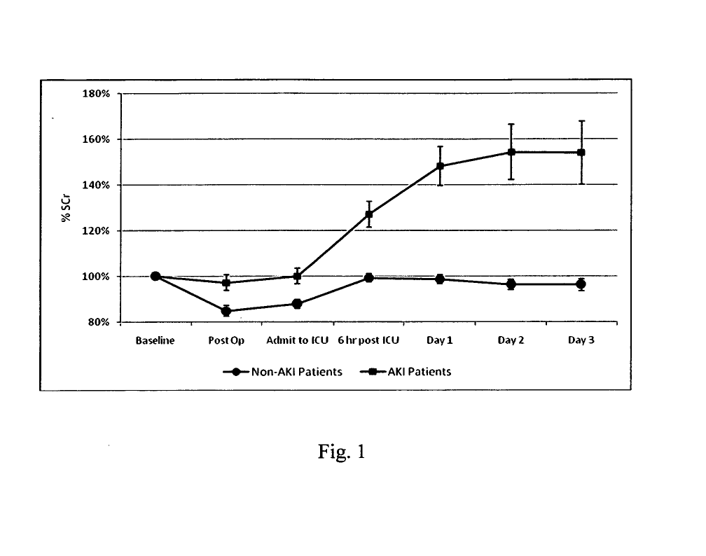

The results are shown in Table 1 and FIGS. 1-3

TABLE 1
Future Development of AKI (as defined above) by Day 2 post
surgery AUC* for ROC** Curves & Sensitivity/Specificity
at indicated time points.

| | AUC | Sensitivity | Specificity |
|---|---|---|---|
| Urinary Pi GST Post Op | 0.679 | 63.6% | 72.2% |
| % SCr Post Op | 0.5 | 0.0% | 100.0% |
| % SCr ICU Admit | 0.5 | 0.0% | 100.0% |
| % SCr 6 hr post ICU | 0.56 | 12.0% | 100.0% |
| % SCr Post Op Day 1 | 0.72 | 44.0% | 100.0% |
| ΔSCr Post Op | 0.545 | 9.1% | 100.0% |
| ΔSCr ICU Admit | 0.538 | 7.7% | 100.0% |
| ΔSCr 6 hr post ICU | 0.76 | 52.0% | 100.0% |
| ΔSCr Post Op Day 1 | 0.84 | 68.0% | 100.0% |

*Area under Curve.
*Receiver Operating Characteristic.

FIG. 1. shows the percentage change in SCr from pre-operative baseline values for non-AKI patients (--) and AKI patients (-▪-). As shown in FIG. 1, the percentage change in SCr does not increase until after the patients have been admitted to ICU. However, as AKI is defined as an increase in SCr of 1.5 fold from baseline, detection of AKI by SCr does not occur until Day 2.

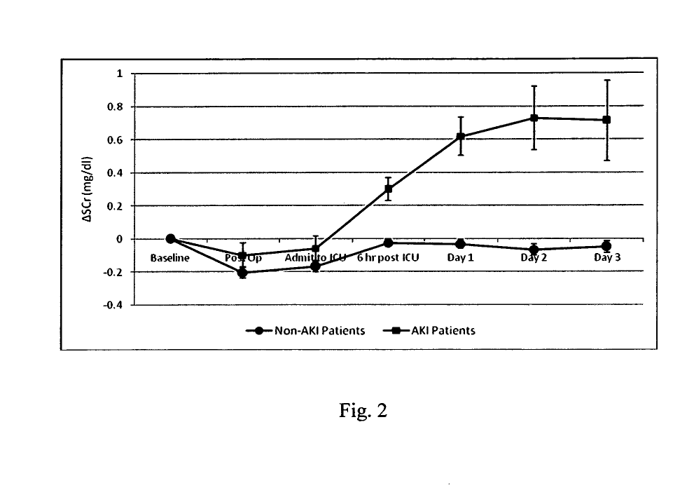

FIG. 2. shows the change in absolute value of SCr from pre-operative baseline values for non-AKI patients (--) and AKI patients (-▪-). As shown in FIG. 2, a significant increase in SCr concentration does not occur until 6 hours post ICU in AKI patients. As the definition of AKI is an absolute increase in SCr of more than or equal to 0.3 mg/dl, AKI would not be diagnosed until after 6 h Post ICU.

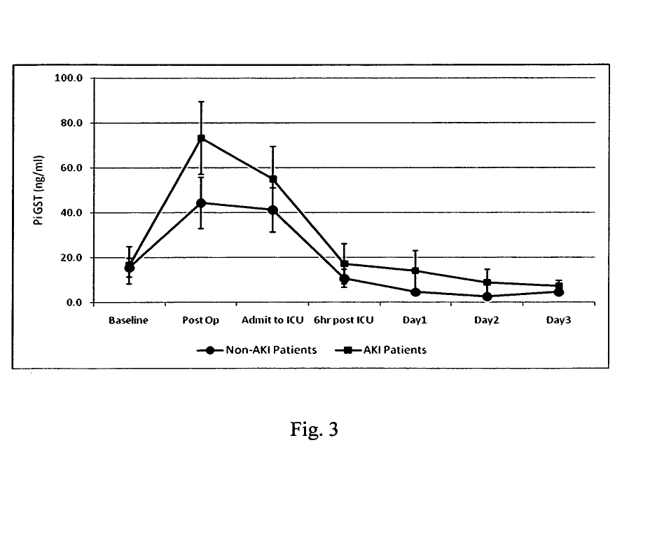

FIG. 3. shows urinary πGST levels following CT surgery for non-AKI patients (--) and AKI patients (-▪-). As shown in FIG. 3, a significant increase in πGST concentration is observed in Post Op. This indicated that patients could be diagnosed with AKI before they are admitted to ICU. Although an increase in πGST is observed in non-AKI patients, it is significantly lower than AKI patient πGST levels, allowing diagnosis of AKI.

Significantly elevated levels of πGST are detected post-op, namely at zero hours.

Analysis of the data shows that πGST is a good early indicator of patients that will develop AKI by day 2 post surgery.

### EXAMPLE 2

### Use of πGST as a Biomarker for a Requirement for RRT Patients Undergoing CT Surgery.

A study was carried out on the 68 patients, the subject of Example 1, using the same methodology for the detection of SCr and πGST.

Seven patients out of the 68 patients tested required RRT. The results are shown in Table 2.

TABLE 2

| | Baseline | Creatinine | Hours in | |
|---|---|---|---|---|
| | Creatinine | at RRT | ICU prior | |
| | (mg/dL) | (mg/dL) | to RRT | Indication |
| 1 | 5.03 | 5.4 | 25.3 | Refractory Hyperkalemia (6.0), Oliguria |
| 2 | 1.49 | 3.48 | 51.2 | Anuria, Elevated creatinine, Shock |
| | 1.36 post-op | | | |
| 3 | 1.3 | 1.42 | 21.6 | Volume overload, Hypoxia, Oliguria, Hemodynamic instability |
| | | | | *AKI not diagnosed using current SCr measures* |
| 4 | 1.2 | 3.79 | 26.8 | Lactic Acidosis Oliguria, Shock, Elevated creatinine |
| 5 | 0.99 | 1.28 | 3 | Lactic Acidosis Anuria, Shock, |
| | | | | *AKI not diagnosed using current SCr measures* |
| 6 | 1.19 | 1.74 | 5.3 | Anuria, Shock (3 pressors), Volume overload. Acidosis |
| 7 | 1.66 | 2.8 | 81 | Volume overload, pulmonary edema. Shock |

The time point at which patients requiring RRT would be first diagnosed is shown in Table 3.

TABLE 3

| | SCr increase ≧150% | | | | | SCr increase ≧0.3 mg/dL | | | | | Pi GST >90 ng/ml | | | | |
|---|---|---|---|---|---|---|---|---|---|---|---|---|---|---|---|
| | Post- | Admit to | 6 hr post | | | Post- | Admit to | 6 hr post | | | Post- | Admit to | 6 hr post | Day | Day |
| | op | ICU | ICU | Day 1 | Day 2 | op | ICU | ICU | Day 1 | Day 2 | op | ICU | ICU | 1 | 2 |
| 1 | | | | | | | | Pos | | | Pos | | | | |
| 2 | | | | Pos | | | | | Pos | | Pos | | | | |
| 3 | | | | | | | | | | | Pos | | | | |
| 4 | | | Pos | | | | | Pos | | | Pos | | | | |
| 5 | | | | | | | Pos | | | | * | Pos | | | |
| 6 | | | | Pos | | | Pos | | | | Pos | | | | |
| 7 | | | | | | | | | | Pos | | | | | |

* No sample available for testing

Table 4 shows the sensitivity and specificity of πGST to detect RRT as summarised therein.

TABLE 4

| | | | |
|---|---|---|---|
| | Time | | |
| Cut off | point | No RRT | RRT |

As determined by Pi GST concentration

| | | | | | |
|---|---|---|---|---|---|
| # patients/group | 90 ng/ml | Post-op | No AKI | 43 | 1 |
| | | | AKI | 9 | 5 |

| | |
|---|---|
| Sensitivity: 83% | Specificity: 83% |

As determined by % SCr increase

| # patients/group | 50% | Post-op | No AKI | 49 | 5 |
|---|---|---|---|---|---|
| | | | AKI | 0 | 0 |
| Sensitivity: 0% | | | Specificity: 100% | | |
| # patients/group | 50% | Admit to | No AKI | 60 | 7 |
| | | ICU | AKI | 0 | 0 |
| Sensitivity: 0% | | | Specificity: 100% | | |
| # patients/group | 50% | 6 hr post | No AKI | 56 | 5 |
| | | ICU admit | AKI | 2 | 1 |
| Sensitivity: 17% | | | Specificity: 97% | | |
| # patients/group | 50% | Day 1 | No AKI | 51 | 4 |
| | | | AKI | 8 | 3 |
| Sensitivity: 43% | | | Specificity: 86% | | |
| # patients/group | 50% | Day 2 | No AKI | 55 | 4 |
| | | | AKI | 4 | 3 |

| | |
|---|---|
| Sensitivity: 43% | Specificity: 93% |

As determined by increase of 0.3 mg/dl in SCr

| # patients/group | 0.3 mg/dl | Post-op | No AKI | 47 | 5 |
|---|---|---|---|---|---|
| | | | AKI | 2 | 0 |
| Sensitivity: 0% | | | Specificity: 96% | | |
| # patients/group | 0.3 mg/dl | Admit to | No AKI | 58 | 5 |
| | | ICU | AKI | 2 | 2 |
| Sensitivity: 29% | | | Specificity: 97% | | |
| # patients/group | 0.3 mg/dl | 6 hr post | No AKI | 47 | 3 |
| | | ICU admit | AKI | 11 | 3 |
| Sensitivity: 50% | | | Specificity: 81% | | |
| # patients/group | 0.3 mg/dl | Day 1 | No AKI | 41 | 3 |
| | | | AKI | 18 | 4 |
| Sensitivity: 57% | | | Specificity: 69% | | |
| # patients/group | 0.3 mg/dl | Day 2 | No AKI | 45 | 3 |
| | | | AKI | 14 | 4 |

| | |
|---|---|
| Sensitivity: 57% | Specificity: 76% |

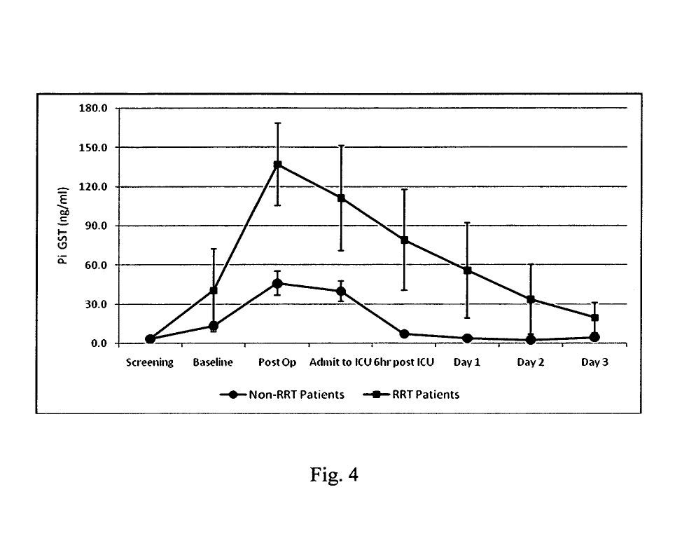

The results are also depicted in FIGS. 4-6.

FIG. 4. shows the variation in urinary πGST post CT surgery for non-RRT patients (--) and RRT patients (-▪-). It will be noted that the πGST level of RRT Patients is significantly higher than non-RRT Patients at the Post Op time point. FIG. 4 shows a concentration of 135 ng/ml is reached, which is considerably higher than AKI patients shown in FIG. 3 (75 ng/ml). This indicates severe AKI and that RRT is required.

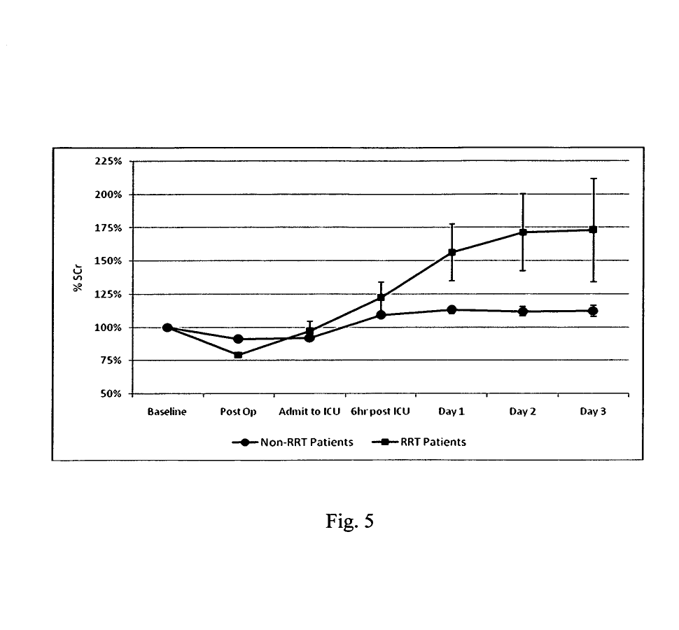

FIG. 5. depicts the variation in percentage SCr from baseline post CT surgery for non-RRT patients (--) and RRT patients (-▪-). FIG. 5 shows that the percentage change of SCr above baseline is not significantly elevated above 1.5 fold increase (AKI) until Day 2. This indicates that the earliest diagnosis that RRT is required using this technique would be two days following surgery.

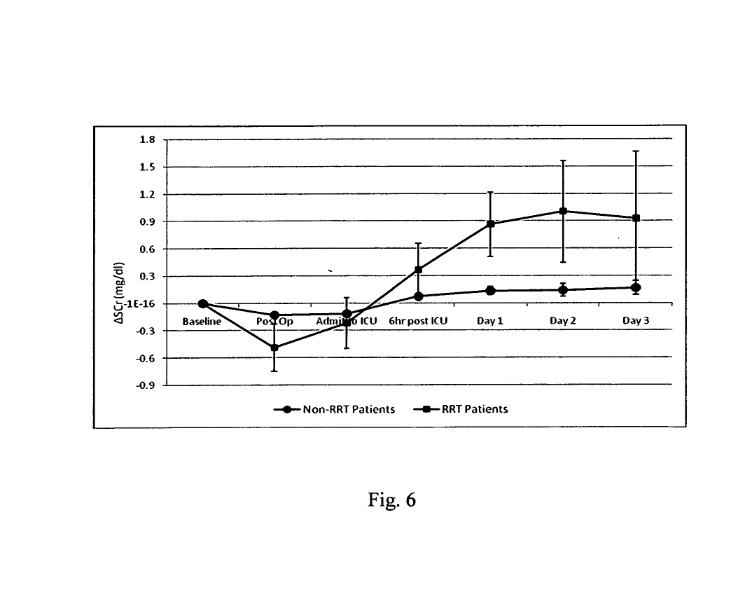

FIG. 6. shows the variation in SCr from baseline post CT surgery for non-RRT patients (-⊙-) and RRT patients (-▪-). It will be noted from FIG. 6 that the absolute change in SCr peaked at Day 2, post surgery. At 6 h post ICU a level of 0.3 mg/dl was reached which indicates AKI. Higher concentrations of SCr were measured at Day 1 and Day 2 indicating severe AKI and a need for RRT. Using this method, RRT would not begin until one day after surgery.

From FIG. 3 and FIG. 4 a relationship is evident between the concentration of πGST and the damage incurred to the patients' kidneys. A πGST concentration of 300%-500% relative to baseline indicates AKI. However, a πGST concentration greater than 500% indicates severe AKI and a requirement for RRT.

The results show that πGST is a very good early indicator of patients that will require RRT undergoing and post CT surgery.

The above Examples show that πGST can be used to detect AKI and a requirement for RRT earlier than with current biomarkers used to detect an abrupt reduction in kidney function due to renal ischemia intraoperatively or post CT surgery, with the attendant advantages.

1. A method for the early identification and prediction of an abrupt reduction in kidney function in a patient undergoing cardiothoracic (CT) surgery, which method comprises contacting a urine sample from the patient with a capture molecule for a biomarker specific for the distal region of the renal tubule and which biomarker is released from said region when there is damage to said region indicative and predictive of an abrupt reduction in kidney function, the biomarker being detectable as early as intraoperatively, allowing for immediate corrective medical intervention.

2. A method according to claim 1, wherein the biomarker is detectable in the recovery stage post CT surgery.

3. A method according to claim 1, wherein the biomarker is detectable prior to transfer of the patient to the Intensive Care Unit (ICU).

4. A method according to claim 1, wherein the abrupt reduction in kidney function is caused by Acute Kidney Injury (AKI).

5. A method according to claim 1, wherein the abrupt reduction in kidney function results in a requirement for Renal Replacement Therapy (RRT).

6. A method according to claim 1, wherein the biomarker is detected earlier than 2 hours post CT surgery.

7. A method according to claim 1, wherein the biomarker is detected earlier than 2 hours post Cardio-Pulmonary Bypass (CPB).

8. A method according to claim 7, wherein the biomarker is detected at zero hours post CT surgery or CPB.

9. A method according to claim 1, wherein the biomarker is pi glutathione S transferase (πGST).

10. A method according to claim 1, wherein the biomarker is detected by immunoassay.

11. A method according to claim 9, wherein the capture molecule is an antibody to πGST.

12. A method according to claim 9, wherein the biomarker is detected enzymatically.

13. A method according to claim 1, wherein the biomarker is detected by a point-of-care assay.

14. πGST for use as a biomarker for the early identification and prediction of an abrupt reduction in kidney function in a patient undergoing CT surgery.

15. πGST for use as a biomarker for AKI post CPB.

16. πGST for use as a biomarker for the identification and prediction of patients undergoing CPB requiring RRT.
